(19) United States
(12) Patent Application Publication
(10) Pub. No.: US 2025/0327119 A1
(43) Pub. Date: Oct. 23, 2025

(54) METHOD FOR DETECTING THE PRESENCE OF A BIOLOGICAL SPECIES OF INTEREST BY ITERATIVE REAL-TIME SEQUENCING

(71) Applicant: BIOMERIEUX SA, Marcy l'Etoile (FR)

(72) Inventor: Sébastien Hauser, Voreppe (FR)

(73) Assignee: BIOMERIEUX SA, Marcy l'Etoile (FR)

(21) Appl. No.: 18/716,107

(22) PCT Filed: Dec. 14, 2022
(86) PCT No.: PCT/EP2022/085847

§ 371 Date: Nov. 1, 2024

(30) Foreign Application Priority Data
Dec. 15, 2021 (FR) FR2113604

(51) Int. Cl. C12Q 1/6869; C12Q 1/6806; C12Q 1/6809; C12Q 1/689; G16B 20/10; G16B 40/10; G16B 50/30

(52) U.S. Cl. CPC C12Q 1/6869; C12Q 1/6806; C12Q 1/6809; C12Q 1/689; G16B 20/10; G16B 40/10; G16B 50/30

(57) ABSTRACT

A metagenomic analysis of a sample for the purpose of detecting the presence of a species of interest in the sample is carried out iteratively. During each iteration, sequences corresponding to each species of interest are identified and counted. The iterations stop when the presence of a species of interest is confirmed or when a maximum number of iterations have been carried out. The detection of a species of interest can be followed by a more precise characterization of the genome of said species of interest. The characterization implements supplementary iterations.

### TECHNICAL FIELD

The technical field of the invention is that of identifying a biological species of interest by metagenomic analysis, employing a real-time sequencing technique.

### PRIOR ART

Sequencing technologies referred to as “real-time” technologies have recently emerged. These platforms allow instant or short-delay access to the sequences as they are read. Additionally, the quantity of sequences read is no longer limited by the need for mobilization of the nucleic acids on a support, thereby potentially opening access to the entirety of the nucleic acid sequences present in the sample analyzed. One example of a technique is the nanopore sequencing of DNA, as implemented by the MinION and GridION sequencing platforms from Oxford Nanopore Technologies.

Nanopore sequencing of DNA is based on the passage of a molecule comprising an oligonucleotide strand through a nanopore, which forms a channel. When the molecule passes through the channel, a potential difference can be measured on either side of the channel that is dependent on the nature of each base forming the strand. The potential difference allows the five standard bases of DNA or RNA (G, C, T or U) to be differentiated. Hence during passage through the nanopore, the oligonucleotide strand induces a temporal sequence of potential differences, and on this basis the order of the bases forming the strand is determined.

Document WO2021/013900 describes a method for detecting a species of interest, more particularly a bacterium, in a sample. The sample has been admixed beforehand with a control species, in a known quantity. After sequencing, the number of sequences corresponding to the control species is used to quantify a concentration of species of interest or to determine a minimum detectable concentration of the species of interest in the sample. The use of the control species ensures that the method of extraction and possible amplification of the nucleotide sequences is carried out correctly. The quantity of control species introduced into the sample is known and so may be used to quantify a concentration or a minimum detectable concentration of the species of interest.

The inventor has adapted the above-described method to the use of real-time sequencers which deliver their digital nucleotide sequences (also called “reads” and corresponding to DNA or RNA sequences) as the nucleotide fragments in the sample are read. This is because these fragments produce an analytical result more rapidly, in several hours, for example, or even several tens of minutes.

### DISCLOSURE OF THE INVENTION

A first subject of the invention is a method for detecting a biological species of interest potentially present in a sample, the biological species of interest having a known or partially known genome, the sample comprising a mixture of different biological species, the method comprising the following steps:
a) adding a control species to the sample, the control species having a known genome, the control species being added in a known concentration to the sample;
b) extracting nucleic acids from the sample;
c) sequencing a portion of the nucleic acid sequences extracted in step b), using a real-time sequencer, for a predetermined duration or until a predetermined number of sequencings is obtained;
d) following step c), assigning sequences resulting from step c) to the species of interest and to the control species;
e) updating the quantities of sequences respectively associated with the species of interest and the control species, such that the quantities of sequences associated with the control species and with the species of interest are:
   in a first iteration of step e), the quantities of sequences assigned to the control species and to the species of interest in step d), added to quantities of initial sequences;
   in each reiteration of step e), the quantities of sequences

$$( {n}_{SOI}^{i} , {n}_{SPC}^{i} )$$

assigned to the control species and to the species of interest in step d), added to the quantities of sequences associated with the control species and with the species of interest resulting from the preceding iteration;
f) comparing the quantity of sequences associated with the species of interest updated in step e) with a threshold of the number of sequences;
   and/or
comparing the quantity of sequences associated with the control species updated in step e) with a control threshold;
g) depending on at least one comparison made in step f), detecting the presence of the biological species of interest in the sample, or proceeding to step h);
h) reiterating steps c) to g) until an iteration stop criterion is reached.

According to one embodiment, step d) of each iteration is carried out for a predetermined duration or until a predetermined number of sequences are sequenced.

The method may be such that:
in step f) of an iteration, when the quantity of sequences associated with the species of interest is greater than the threshold of the number of sequences;
in step e) of the iteration, when the quantity of sequences associated with the control species is strictly greater than zero;
   step g) comprises an estimation of a concentration of the species of interest, depending:
on the quantity of sequences associated with the species of interest at the last updating (i.e., at the last step e);
on the quantity of sequences associated with the control species at the last updating;
on the added concentration of the control species.

The method may be such that:
in step f) of an iteration, when the quantity of sequences associated with the species of interest is greater than the threshold of the number of sequences;
in step e) of the iteration, when the quantity of sequences associated with the control species is zero;
   during step g), the concentration of the species of interest is deemed to be greater than the concentration of the control species.

The method may be such that:
in step f) of an iteration, when the quantity of sequences associated with the species of interest is less than the threshold of the number of sequences;
in step f) of the iteration, when the quantity of sequences associated with the control species is greater than the control threshold;
   during step g), if the quantity of sequences associated with the species of interest is zero, the concentration of the species of interest is deemed to be zero.

The method may be such that:
in step f) of an iteration, when the quantity of sequences associated with the species of interest is less than the threshold of the number of sequences;
in step f) of the iteration, when the quantity of sequences associated with the control species is greater than the control threshold;
   during step g), if the quantity of sequences associated with the species of interest is non-zero, step g) comprises an estimation of a concentration of the species of interest, depending:
on the quantity of sequences associated with the species of interest;
on the quantity of sequences associated with the control species;
on the added concentration of the control species.

The concentration of the species of interest may be estimated from a ratio:

$$\frac{{N}_{SOI}^{i}}{{N}_{SPC}^{i}} \times \frac{{L}_{SPC}}{{L}_{SOI}} \times {C}_{SPC}$$

in which:
   L_{SPC} and L_{SOI} are respectively the genome lengths of the control species and of the species of interest;

$${N}_{SOI}^{i} and {N}_{SPC}^{i}$$

are respectively the quantities of sequences respectively associated with the species of interest and with the control species resulting from the last updating;
   C_{SPC} is the concentration of the control species added to the sample.

The method may be such that, after the iteration stop criterion is reached,
if the quantity of sequences associated with the species of interest is less than the threshold of the number of sequences;
and if the quantity of sequences associated with the control species is less than the control threshold;
the method generates information to the effect that neither the presence nor the absence of the species of interest can be confirmed.

According to one possibility, the method comprises taking into account a decision threshold, the method aiming to compare a concentration of the species of interest to the decision threshold, the method being such that in step a), the concentration of the control species is between 0.01 times and 100 times the decision threshold.

According to one possibility, in step h), the iteration stop criterion is reached when:
the quantity of sequences associated with the species of interest exceeds the threshold of the number of sequences;
and/or the quantity of sequences associated with the control species exceeds the control threshold;
and/or the cumulative duration of steps c) reaches a predetermined maximum duration;
and/or the cumulative number of sequencings in steps c) reaches a predetermined maximum number;
and/or the number of iterations reaches a predetermined maximum number of iterations.

According to one embodiment, following detection of the presence of the species of interest in the sample, the method comprises the following steps:
i) sequencing a portion of the nucleotide sequences extracted in step b), using a real-time sequencer, for a predetermined duration or until a predetermined number of sequencings is obtained;
j) following step i), assigning sequences resulting from step i) to the species of interest detected;
k) updating the quantities of sequences associated with the species of interest, such that the quantities of sequences associated with the species of interest are:
   in a first iteration of step k), the quantity of sequences assigned to the species of interest detected in step j), added to the quantity of sequences resulting from the last iteration of steps c) to g);
   in each reiteration of step k), the quantity of sequences assigned to the species of interest in step j), added to the quantity of sequences resulting from the preceding iteration of step k);
l) reiterating steps i) to k) until an iteration stop criterion is reached.

Steps i) to k) may be reiterated until a predetermined number of iterations is reached.

According to one possibility:
during each iteration of steps i) to k), following step k), the method comprises the determination of a depth of sequencing of the biological species of interest, the depth of sequencing corresponding to a ratio between the cumulative length of the sequences associated with the species and the length of the genome of the species;
steps i) to k) are reiterated until a predetermined depth of sequencing is reached.

According to one embodiment, following the stopping of the iterations of steps i) to k), the detection of a typical sequence of the genome of the species of interest, the typical sequence comprising an antibiotic resistance marker or a virulence marker for the species of interest. The sequencer is preferably configured to carry out successive sequencings of different sequences of nucleic acids, one after another, the sequencing of each sequence being deemed to be carried out in real time.

A second subject of the invention is a device for metagenomic analysis of a sample, comprising:
a fluidic chamber intended to receive the sample;
a sequencer configured to carry out real-time sequencing of the sample;
a processing unit, comprising a bioinformatics module programmed to assign sequences resulting from the sequencer to a species, and a control module programmed to implement steps c) to h) of a method according to the first subject of the invention.

A third subject of the invention is a recording medium, readable by computer or downloadable, comprising the instructions for implementing steps c) to h) of a method according to the first subject of the invention.

The invention will be better understood on reading the disclosure of the exemplary embodiments presented, in the remainder of the description, with reference to the figures listed below.

### FIGURES

FIGS. 1 and 2 schematically show the principal steps of a method according to the invention.

FIG. 1 relates to a phase for detection of a species of interest.

FIG. 2 relates to a phase for characterization of each species of interest detected in the detection phase.

FIG. 3 schematically shows a device configured for implementing the invention.

### DISCLOSURE OF PARTICULAR EMBODIMENTS

The objective of the method is to be able to detect the presence of a biological species of interest SOI in a sample. The acronym SOI signifies “species of interest”. In the event of detection, the process may permit absolute quantification of the species of interest SOI, so as to allow comparison with a decision threshold SD.

By biological species, what is meant is a microorganism, for example a bacterium, or a virus, a fungus, an archaebacterium, an amoeba, a protist, or a microalga. A biological species may also be a cell or any other object or entity comprising a sequenceable nucleic acid.

When the sample is obtained from a human or animal organism, the biological species of interest may be a pathogenic species or a species identified as having an impact on the health or the functioning of the human or animal body, such as for example intestinal bacterial species, or bacteria affecting the efficacy of an anti-cancer treatment. When the sample is obtained by sampling from an industrial process or from the environment, the biological species of interest may be a species considered to be a contaminant, or a species of interest having an importance in an industrial process or in the environment, and the presence or concentration of which it is desired to police.

The species of interest exhibits a known or partially known genome. The genome, or its known portion, is made up of sequences, called sequences of interest.

The method may address a plurality of species of interest simultaneously. The term “a species of interest” should therefore be interpreted as signifying at least one species of interest.

The decision threshold SD is a threshold that makes it possible to characterize a load of the biological species of interest, of a microorganism for example, depending on the targeted application. It is for example set in light of a regulatory, or sanitary or industrial, limit. For example, when the application is used in assistance with clinical diagnosis, the biological species of interest being a bacterium, the decision threshold may be a concentration below which the presence of the bacterium corresponds to a normal presence, i.e., a non-pathological development, and above which the presence of the bacterium is deemed to be pathological, and for example to correspond to an infection. When the invention is applied to an industrial process, the decision threshold corresponds to a pass value, such that above the decision threshold the sample is considered not to pass, and below the detection threshold the sample is considered to pass. Whatever the application, when the concentration of the biological species of interest is higher than or equal to the decision threshold, it is defined as being critical. In certain applications, for example in the manufacture of fermented products, a concentration of biological species of interest may be considered to be critical if it is lower than a decision threshold, the latter corresponding to a minimum acceptable concentration of the biological species.

The sample is generally a sample taken from the environment or from a dead or living organism, or even from an agrifood or manufactured product. The sample may also have been taken from an industrial facility, for purposes of process control. Thus the sample comprises various biological species, not having the same genome. In particular, when the sample results from sampling of an organism, for example a human or animal organism, the sample comprises a significant or even predominant quantity of cells originating from the organism from which the sample has been taken. The genomes of human or animal organisms have a size that is 1000 to 100 000 times larger than the genomes of prokaryotic organisms. In addition, the sample generally comprises biological species that are naturally present in the sample, and not liable to result in a pathology or a critical contamination. For example, when the sample is a bronchoalveolar sample, it comprises a bacterial flora naturally present in the lungs. When the sample is a stool sample, it comprises a bacterial flora naturally present in the digestive tract. Hence, when the biological species of interest is a bacterium or a virus, the nucleic acids of the biological species of interest may be a minority of the nucleic acids in the sample.

The sample comprises what are called “matrix” species, which are endogenous to the sample, and which are liable to mask metagenomic information relative to the biological species of interest. For example, when the sample is taken from a yoghurt, from a piece of meat or from a vaccine, it comprises matrix species that are representative of these media. In the case of sampling from an organism, the matrix comprises the constituent cells of the organism.

An important aspect of the invention is that the presence of the species of interest in the sample is detected using a prior-art real-time sequencer. A sequencer of this kind allows exploitable data to be obtained that relate to the sequences present in the sample, more rapidly than earlier sequencers. The real-time sequencer is configured to successively collect signals representative of successive detections of bases in a single sequence. Accordingly, a real-time sequencer is configured to generate a sequence in real time, as the sequence is decoded. Sequencings of different sequences present in the sample are carried out successively, each sequence being sequenced one after another. This does not rule out the possibility of certain sequences undergoing simultaneous sequencings, in parallel. For example, when the sequencer is a nanopore sequencer, each nanopore “reads” a number of different sequences in parallel. Each nanopore may read a number of different sequences successively.

Another advantage of such a sequencer is the possibility of carrying out real-time analysis of sequences, and the quantity of sequences read relates to the duration of the sequencing. Depending on the sequencing results available, sequencing may be continued or halted, providing a time gain and high flexibility of usage. The sequencer may be of nanopore type, as described in the prior art. More generally, the invention relates to the use of a sequencer configured to generate sequences in real time, these sequences being identified after they are sequenced. Hence the sequencer is configured to sequentially generate a list of sequences identified, these identified sequences being denoted by the term “reads”. The lists of reads are generated at regular intervals, for example every n minutes, n being for example 10. It is also possible to parameterize the quantity of sequences read in each list. The quantity of sequences (or reads) in each list may for example be 4000.

Similarly to the situation in patent application WO2021/013900, one of the objectives of the invention is to evaluate the extent to which a metagenomic analysis is exploitable. It is necessary in particular to evaluate the conformity of the collective steps from the preparation of the sample, sampling excepted, up to the bioinformatic analysis of the sequencing data. For this purpose, a control species, denoted SPC, an acronym for Sample Processing Control, is added to the sample. One function of the control species is to enable policing of the effective progress of the steps of nucleic acid extraction and of sequencing, described hereinafter. The control species SPC may be a known biological species, the genome of which is also known, preferably in its entirety. The control species SPC may be a natural biological species. It may also be an artificial species, for example an encapsidated RNA (ribonucleic acid). Preferably, the control species SPC is not initially present in the sample taken, or if so in a negligible quantity. Preferably, the content of control species SPC initially present in the sample, i.e., present before the addition, is preferably at least 10 times lower, or preferably at least 100 or 1000 times lower, than the concentration C_{SPC} of the control species SPC added to the sample. The control species SPC may for example be a bacterium. It is important for the concentration C_{SPC} of the control species SPC added to be controlled.

The control species may be chosen taking into account the aspects listed below:
The control species must preferably differ from the organisms naturally present in the sample, or endogenous organisms, and from the sought-after species of interest: thus, the bioinformatic tool is able to accurately identify the sequences from the sequencing of the SPC.
The quantity of sequences assigned to the control species, during the sequencing, must be sufficient to be able to be detected correctly, though without masking the useful information, corresponding to the sequences of the biological species of interest. In other words, the control species is preferably detectable by high-throughput sequencing, while not being preponderant in the sample. In particular, when the desire is to determine a positiveness (concentration of the species above the decision threshold) or a negativeness (concentration of the species below the decision threshold), it is preferable for the control species to exhibit at least one of the following characteristics:
   The size of its genome is preferably similar, or at least comparable, to the size of the genome of the biological species of interest. More particularly, the size of the genome of the control species is between 0.1 times to 10 times the size of the genome of the biological species of interest.
   The concentration C_{SPC} of the control species may be set depending on the decision threshold. The concentration C_{SPC} of the control species SPC added may for example be between 0.001 times and 1000 times, and preferably between 0.01 and 100 times, the decision threshold.
   The nucleic acids of the control species SPC undergo a similar treatment to the nucleic acids of the species of interest in the steps of preparing the sample, of extracting and of sequencing, and preferably:
      the percentage of GC (guanine, cytosine) bases is preferably close to the percentage of GC bases of the biological species of interest; by close to, what is meant is between 75% and 125%, and preferably between 80% and 120%.
      The control biological species, when the biological species of interest is a bacterium, preferably comprises an intact cell wall or a membrane, or, when the biological species of interest is a virus, preferably comprises a protein shell. This condition also allows the steps of lyzing or of extracting nucleic acids of the biological species of interest to be monitored.
Preferably, the nucleotide sequences of the control species do not contain genomic markers, such as for example markers of resistance to antibiotics, or virulence markers, so as not to cause the results of a potential test of sensitivity to antibiotics to be corrupted by the presence of such markers in the genome of the biological species of interest. Preferably, the nucleotide sequences of the control species do not contain any other gene of clinical or industrial interest and the presence of which is liable to be checked for.
The control species is preferably easily manipulatable, being in particular:
   harmless to humans or to the environment;
   and/or resistant to heat treatments such as freeze-drying or freezing, thereby facilitating storage.
The control species must not form spores, or if so only marginally.
The control species must have a sensitivity to lysis that is close to that of the biological species of interest.
The control species may take the form of balls, each ball comprising a calibrated concentration of control biological species in freeze-dried form.

It will be noted that a single control species SPC may be used, or that a plurality of control species, of various types, may be used.

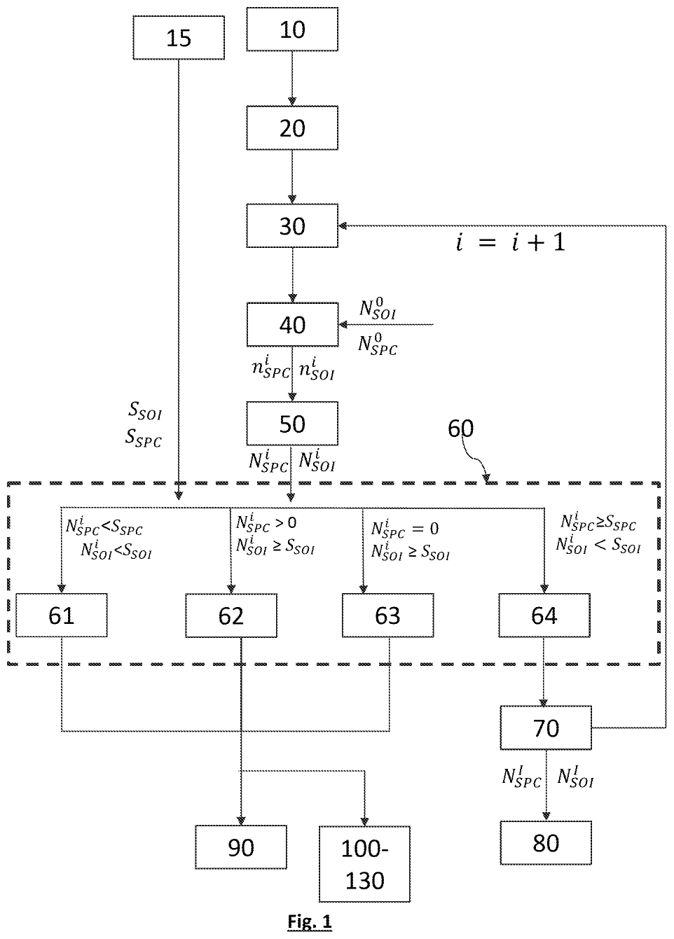

The method first comprises a phase of detecting a species of interest, with the steps described below, in conjunction with FIG. 1.

Step 10: Taking the sample.

In this example, the sample is taken from a living human organism, for purposes of assisting with diagnosis. However, the invention is not limited to an application in the realm of living things. The sample may be taken from a natural, industrial or hospital environment, so as to verify a conformity with respect to a decision threshold.

Step 20: Adding the control species.

The added concentration C_{SPC} of the control species SPC is preferably known with precision, for example with the same precision as that desired for the quantification of the one or more species of interest. Specifically, it may allow, provided that certain conditions are met, the concentration of biological species of interest in the sample to be quantified, the control species then forming a calibrator. The term “added concentration” designates the concentration of the control species in the sample due to the addition of the control species.

In the description of steps 30 to 60, the addition of a single type of control species to the sample is taken as the basis, by way of advantageous example. The control species then performs the function of quality control in the steps of the metagenomic analysis, and the function of a calibrator, allowing possible quantification of the concentration of the biological species of interest.

At the end of step 20, there is an added concentration C_{SPC} of the control species in the sample. The added concentration C_{SPC} of the control species is preferably expressed in GEq/ml (genome equivalent per mL).

It may be noted that the clinical detection thresholds in bacteriology (e.g., the threshold above which an infection is diagnosed) are generally expressed in CFU/mL (colony-forming units), but the proposed quantification technique allows nucleic acids to be quantified without information on the genome equivalence. It is therefore recommended that, for the SPC, the correlation is determined between the number of units forming a colony, by culturing, for example, on a gel medium, and the equivalence in terms of number of genomes, by quantitative PCR, for example. It has been established, for example, that in a Bioball® B. subtilis strain ATCC 19659 BioBall® MultiShot 10E8 (bioMérieux, catalog #416721), one colony-forming unit was equivalent to one genome.

The concentration added may be defined as a function of the decision threshold SD. The added concentration C_{SPC} of the control species is preferably equal to the decision threshold SD, or close to the decision threshold SD, for example to within about ±50%. Hence 0.1 SD≤C_{SPC}≤10 SD. The effect of the added concentration C_{SPC} of the control species is described below.

Step 30: Lyzing and extracting nucleic acids.

In this step, the cells of the sample, and notably the cells of the biological species of interest and of the control species, undergo lysis, in order to allow their DNA to be extracted. Various strategies may be envisioned:
The lysis may be parameterized to preferentially target the biological species of interest. In such a scenario, the control species must have the same sensitivity to lysis as the biological species of interest, or a sensitivity to lysis that is considered equivalent.
The lysis may include a first lysis, intended to lyze essentially cells other than the species of interest. Such a first lysis may for example be envisioned when the biological species of interest is in a very small minority with respect to the cells of a matrix constituting the sample. Following the first lysis, the nucleic acids released are removed, and then a second lysis is carried out, targeting the biological species of interest. In such a scenario, the control species is preferably resistant to the first lysis, and not resistant to the second lysis.

Following the lysis, DNA is extracted from the sample in accordance for example with the extraction method described in WO2014/114896.

Step 40: sequencing

The DNA extracted from the sample undergoes sequencing by means of a sequencer which allows real-time access to the sequences read—for example, a nanopore sequencer. It may for example be a MinION® sequencer from Oxford Nanopore Technologies. The sequencer is connected to a dedicated fluidic chamber, comprising the nanopores. The sample is introduced into the fluidic chamber. The sequencer is connected to a processing unit for analyzing the sequences identified. The processing unit comprises a microprocessor, configured to execute instructions from a piece of bioinformatics software, to identify the sequences analyzed. The processing unit is, for example, a MinIT computer, containing the computing environment dedicated to the sequencing.

The invention is applied in particular to so-called “real-time” sequencers which have the following characteristics:
a capacity to read all of the sequences present in the sample, in contrast to sequencers based on immobilization of the sequences on a support in order to read them, these sequencers therefore sequencing a predefined maximum number of sequences that corresponds to the number of elements for fixing the nucleotide sequences. This implies in particular that the number of sequences produced is proportional to the duration of sequencing.
a capacity to output raw data (e.g., reads or raw signals such as electrical potentials) at the same rate as they are collected, or to output raw data in batches, with a frequency defined per batch of sequences of given size, or regularly at a fixed frequency (for example, one batch of raw data per minute or per ten minutes).

A particular feature of the “real-time” sequencing is that the RNA or DNA sequence reading results are available in real time at the output from the sequencer, or in quasi-real time. Quasi-real time means that the reads (or the raw signals measured such as electrical potentials) are output from the sequencer in batches at the end of a duration very much less than the total duration needed for the sequencing of the acids present in the sample, or very much less than the total duration of consumption of the reagents needed for the sequencing. Each batch may therefore be produced in a few minutes or a few tens of minutes. The real-time sequencer has the capacity to produce sequences (reads) in real time. However, it is generally expected that a certain number of sequences have been decoded, in a file generated at regular intervals. As an example, the Oxford Nanpore MinION Sequencer by default generates, in quasi-real time, “.fast5” files in batches of 4000 sequences of potential differences, which can be converted in quasi-real time by a computer into .fastq files in batches of 4000 reads. The .fast5 extension denotes raw data files. The .fastq extension denotes files which store sequences, and the associated quality scores. This allows the method to be implemented interactively, enabling sequencing to be stopped or continued depending on the results obtained.

The sequencing may in particular be carried out iteratively, with each iteration corresponding to a predetermined number (or batch) of sequences or to sequencing carried out over a predetermined duration. During each iteration, following the sequencing, sequences are potentially assigned to the control species SPC or to the biological species of interest SOI, or to each biological species of interest or to other species present in the sample.

Each iteration has a corresponding rank i. i is an integer between 1 and I, I corresponding to the maximum number of iterations described in conjunction with step 70, or to a maximum number of sequences (reads) obtained. During each iteration, the sequencing assigns numbers of sequences

$${n}_{SOI}^{i} and {n}_{SPC}^{i}$$

respectively to the species of interest SOI and to the control species SPC.

The steps which follow are described in conjunction with a species of interest SOI. They may be implemented with a plurality of different species of interest.

Step 50: Updating the number of sequences.

During this step, the numbers of sequences respectively assigned, in step 40, to each biological species of interest and to the control species are respectively at the number of sequences

$${N}_{SOI}^{i - 1} , {N}_{{SPC}^{1}}^{i - 1}$$

respectively associated with the biological species of interest SOI and with the control species SPC in the preceding iteration. This step corresponds to the updating of the number of sequences respectively associated with the species of interest SOI and with the control species SPC, according to the expressions:

$${N}_{SOI}^{i} = {N}_{SOI}^{i - 1} + {n}_{SOI}^{i} \qquad ( 1 )$$

$${N}_{SPC}^{i} = {N}_{SPC}^{i - 1} + {n}_{SPC}^{i} \qquad ( 2 )$$

where n_{SOI}ⁱ and n_{SPC}ⁱ are the numbers of sequences respectively assigned to the species of interest and to the control species in step 40 of the iteration i. The notation nⁱ corresponds to a number of sequences assigned to a species in the iteration i, whereas the notation Nⁱ corresponds to a total number of sequences associated with a species for the entirety of the iterations up until the iteration i.

In the first iteration (i=1), consideration is given to the predetermined initial values

$${N}_{SOI}^{0} and {N}_{SPC}^{0} ,$$

which are generally zero:

$${N}_{SOI}^{0} = {N}_{SPC}^{0} = 0 .$$

According to one possibility, the numbers of sequences

$${N}_{SOI}^{i} or {N}_{SPC}^{i}$$

may be normalized by a reference quantity. The reference quantity may for example be a total quantity Nⁱ of sequences produced during sequencing, up to the current iteration i.

Step 60: Comparisons with thresholds

During this step, the number

$${N}_{SOI}^{i}$$

of sequences associated with each species of interest SOI is compared with a threshold of the number of sequences S_{SOI}, which corresponds to the minimum number of reads associated with the species of interest SOI that is necessary to stop the sequencing and commence the quantitative interpretation relating to the presence of the species of interest in the sample. The threshold of the number of sequences corresponds to a threshold of the number of sequences, or threshold of the number of reads, which are associated with the species of interest.

The number

$${N}_{SPC}^{i}$$

of sequences associated with the control species SPC is compared with a control threshold S_{SPC}, which corresponds to the minimum number of reads associated with the SPC for stopping the sequencing when the number of reads associated with each of the SOI does not reach the threshold of the number of sequences S_{SOI}.
When

$${N}_{SOI}^{i} < {S}_{SOI} and {N}_{SPC}^{i} < {S}_{SPC} :$$

cf. step 61;
When

$${N}_{SOI}^{i} \geq {S}_{SOI} and {N}_{SPC}^{i} > 0 :$$

cf. step 62;
When

$${N}_{SOI}^{i} \geq {S}_{SOI} and {N}_{SPC}^{i} = 0 :$$

cf. step 63;
When

$${N}_{SOI}^{i} < {S}_{SOI} and {N}_{SPC}^{i} \geq {S}_{SPC} :$$

cf. step 64;

The determination of the threshold of the number of sequences and of the control threshold is described below.

Step 61

During this step, no conclusion can be drawn as to the presence of the species of interest SOI in the sample. A renewed iteration of steps 40 to 60 is required, unless an iteration stop criterion is reached, as described in conjunction with step 70.

Step 62: During this step, it is possible to conclude the presence of the species of interest SOI in the sample and to quantify the concentration thereof, for example via the expression:

$${C}_{SOI} = \frac{{N}_{SOI}^{i}}{{N}_{SPC}^{i}} \times \frac{{L}_{SPC}}{{L}_{SOI}} \times {C}_{SPC} \times α \qquad ( 3 )$$

in which:
L_{SPC} and L_{SOI} are respectively the genome lengths of the control species and of the biological species of interest;
α is a correction factor determined empirically, on the basis of training samples in which the concentration of biological species of interest is known. The correction factor α takes account of the differences in efficacy of the procedure for sequencing the biological species of interest and the control species. As a default, it may be deemed that α=1. This unitary value produces an absolute quantification which is sufficient to determine the positiveness or negativeness of a sample relative to the decision threshold.

When the added concentration is expressed in GEq/mL, the concentration of the biological species of interest is also expressed in the same unit.

Step 63

During this step, the species of interest is considered to be present in the sample, at a concentration greater than the concentration of the control species, but it is not possible to determine the absolute concentration of the SOI.

$${C}_{SOI} >> {C}_{SPC} \qquad ( 4 )$$

Step 64

During this step, if

$${N}_{SOI}^{i} = 0 ,$$

it is possible to conclude that the species of interest SOI is absent from the sample. Otherwise, it is possible to quantify the concentration thereof, using the formula described in step 62.

Step 70: Steps 40 to 60 are reiterated, until an iteration stop criterion is reached. With preference, each iteration i corresponds to a predetermined sequencing duration, which is preferably identical for each iteration, or to a predetermined number of sequences read (number of reads).

The iteration stop criterion is considered to be reached when the conditions leading to steps 62 or 63 or 64 are reached.

The iteration stop criterion may also be a number of iterations equal to a maximum number of iterations I, said number having been predetermined. For example, the iteration stop criterion is considered to be reached when the sequencing duration is greater than or equal to a predetermined duration, 12 hours for example, or when the total number of sequences obtained from the sequencer is greater than a predetermined maximum number.

Step 80 alerting.

Step 80 is carried out if

$${N}_{SOI}^{I} < {S}_{SOI} and {N}_{SPC}^{I} \geq {S}_{SPC} ,$$

i.e., following the last iteration of rank I, the numbers of sequences respectively associated with the species of interest and with the control are respectively lower than the threshold of the number of sequences and than the control threshold. In that event, an alert signal is emitted, indicating that the presence or the absence of the species of interest in the sample cannot be confirmed.

Step 90 stopping

Following step 70 or a possible step 80, the method may be stopped.

According to one possibility, a plurality of species of interest, different from one another, are analyzed simultaneously. When one of the conditions indicated in steps 62, 63 and 64 is encountered, the sequencing may be halted and each species of interest is interpreted according to these criteria.

Optionally, when the presence of a species of interest in the sample has been reported, following steps 62 or 63 or 64, it is possible to have complementary genomic information regarding the species of interest identified. The information in question may in particular be the presence of a specific marker in the genome—for example, an antibiotic resistance marker or a virulence marker. Antibiotic resistance or virulence markers are known.

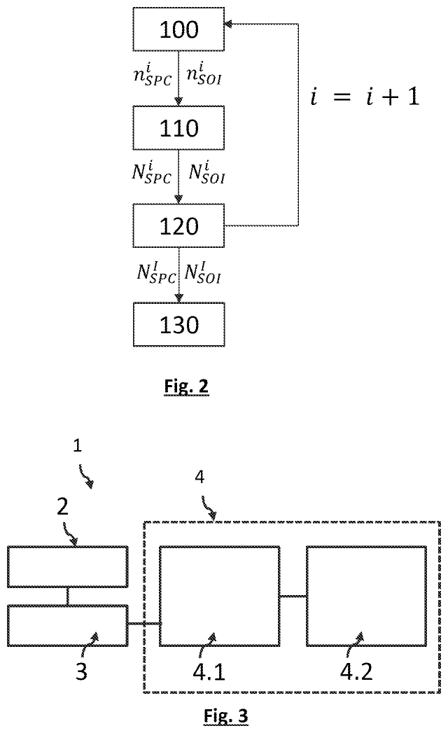

The iterative steps 100 to 130, described hereinafter, in conjunction with FIG. 2, may be implemented in order to confirm the presence of the specific marker in the genome of the species of interest identified. If i′ designates the rank of the iteration in which the iteration stop criterion of step 70 has been reached, steps 100 to 130 are implemented so that i′<i≤I. I corresponds to the rank of the last iteration.

Step 100

This step is similar to step 40. Only the sequences corresponding to each species of interest SOI detected following the iterations of steps 40 to 80 are numbered.

Step 110

This step corresponds to step 50, except that the number of sequences of each species of interest is updated, according to the following expression:

$${N}_{SOI}^{i} = {N}_{SOI}^{i - 1} + {n}_{SOI}^{i} \qquad ( 1 )$$

Step 120: reiteration of steps 100 to 110, unless an iteration stop criterion is reached. The iteration stop criterion may be the reaching of a maximum number of iterations I. As a reminder, the latter may correspond to a maximum sequencing duration or to a maximum number of sequences identified.

According to one possibility, during each iteration, a depth of sequencing of the species of interest SOI is determined. The depth of sequencing corresponds to a ratio between the cumulative length of the sequences associated with the species of interest and the length of the genome of the species of interest. When the depth of sequencing reaches a threshold depth, the iteration stop criterion is reached. The iterations are stopped.

Step 130: Characterizing

During this step, the sequences corresponding to the desired marker are identified and numbered.

Determining the Threshold of the Number of Sequences S_{SOI} and the Control Threshold S_{SPC}

The implementation of the method described beforehand assumes a prior determination:
of the threshold of the number of sequences Sso1, with which the number of sequences of the species of interest

$${N}_{SOI}^{i}$$

is compared in each step 60;
and of the control threshold S_{SPC}, with which the number of control sequences

$${N}_{SPC}^{i}$$

is compared in each step 60.

Estimations were made of the sequencing durations necessary to produce numbers of reads corresponding to the species of interest SOI of respectively 1, 10, 100, 1000 and 10 000, while taking account of different concentrations C_{SOI} of the species of interest SOI in the sample.

The sequencing duration TTR (time to result) is estimated according to the following expression:

$$T T R = \frac{{S}_{SOI} \times {C}_{ADNtot} \times {T}_{fastq} \times {N}_{sample}}{{C}_{SOI} \times {R}_{fastq}} \qquad ( 5 )$$

in which:
S_{SOI} is the threshold of the number of sequences;
C_{ADNtot} is the total concentration of DNA in the sample: this includes the concentrations of the species of interest, of the control species and of the non-target DNA (human DNA and commensal flora);
R_{fastq} is a quantity of reads obtained in each iteration, i.e., in each .fastq file generated. For example, R_{fastq}=4000.
T_{fastq} is a mean sequencing duration for obtaining a .fastq file comprising a quantity of reads equal to R_{fastq};
Nₛₐₘₚₗₑ is the number of samples sequenced simultaneously. Nₛₐₘₚₗₑ corresponds to a proportion of sequenced DNA that may be attributed to the sample. Nₛₐₘₚₗₑ is other than 1 when different samples are sequenced simultaneously. In that case, Nₛₐₘₚₗₑ designates the total quantity of DNA sequenced divided by the quantity of DNA in the sample in question.

Table 1 gives the sequencing duration (in hh:mm) which is calculated depending on the threshold of the number of sequences S_{SOI}, and on the concentration C_{SOI} of species of interest SOI expressed as a function of the decision threshold SD. It has been considered that the control species has been added at a concentration C_{SPC}=SD and that the sum of the human DNA and the commensal flora was equivalent to 100SD. The total quantity of DNA is then equal to C_{ADNtot}=101SD+C_{SOI}. The sample is considered to be sequenced at the same time as three other samples (Nₛₐₘₚₗₑ=4), each of the samples being sequenced with an equivalent quantity of DNA with a sequencer which generates read files comprising R_{fastq}=4000 reads with a frequency of about T_{fastq}=3 minutes.

TABLE 1

| | S_{SOI} | | | | |
|---|---|---|---|---|---|
| C_{SOI} | 1 | 10 | 100 | 1000 | 10 000 |
| 0.0001SD | 50:33 | 505:03 | 5050:03 | 50500:03 | 505000:30 |
| 0.001SD | 5:06 | 50:33 | 505:03 | 5050:03 | 50500:30 |
| 0.01SD | 0:33 | 5:06 | 50:33 | 505:03 | 5050:30 |
| 0.1SD | 0:06 | 0:33 | 5:06 | 50:33 | 505:30 |
| SD | 0:03 | 0:06 | 0:33 | 5:06 | 51:00 |
| 10SD | 0:03 | 0:03 | 0:06 | 0:36 | 5:33 |
| 100SD | 0:03 | 0:03 | 0:03 | 0:09 | 1:03 |
| 1000SD | 0:03 | 0:03 | 0:03 | 0:06 | 0:36 |
| 10 000SD | 0:03 | 0:03 | 0:03 | 0:06 | 0:33 |

In table 1, it is observed that when the concentration C_{SOI} is equal to the decision threshold SD, the value of the threshold of the number of sequences S_{SOI} must not exceed 100 if the desire is to reach the threshold of the number of sequences S_{SOI} in 30 minutes of sequencing. Specifically, the higher the threshold of the number of sequences S_{SOI}, the more the duration of analysis increases. Logically, the higher the threshold of the number of sequences S_{SOI}, the greater too the duration for reaching it. A value S_{SOI}=1 is reached rapidly, even for low concentrations of species of interest. However, the adoption of too low a value of S_{SOI} increases the risk of a false positive and is detrimental to the precision of the quantification.

In addition, too low a value limits the possibility of detection of other species of interest that are potentially present at lower concentrations. For example, the use of a threshold of the number of sequences S_{SOI} of 100 enables detection of another species of interest SOI′, present at a concentration 100 times lower, on the basis of a single read. Therefore, a high threshold of the number of sequences S_{SOI} may be of particular interest if a majority SOI is present at a concentration of more than 100 times SD: this enables the detection of other, minority SOI, denoted SOI′, at a concentration up to 100 times lower than SOI but higher than the decision threshold SD.

On the basis of the results presented in table 1, the conclusion is drawn that the threshold of the number of sequences S_{SOI} is preferably between 5 and 500 or between 50 and 500. When a short duration of analysis is prioritized, a relatively low threshold of the number of sequences S_{SOI} is chosen, of close to 50, or even lower, in awareness of the fact that too low a threshold of the number of sequences is conducive to false positives. When the dynamics of measurement, and the possibility of simultaneous detection of different species of interest, present in different concentrations, are prioritized, a higher threshold of the number of sequences S_{SOI} is chosen, of close to or greater than 500, at the expense of the duration of analysis.

The influence of the control threshold S_{SPC} on the implementation of the method was studied. In the absence of the species of interest, substantial quantities of DNA may be present in the sample. In a clinical sample, the origins of the DNA may be diverse—for example, DNA from the cells of the patient and from the commensal flora. The DNA may also result from an atypical infection: the concentration of genomic DNA from a microorganism other than the SOI may therefore reach quantities very much higher than the decision threshold SD. Table 2, for different concentrations of DNA C_{ADN}, expressed relative to the decision threshold SD, gives the sequencing duration that allows the number of sequences obtained that correspond to the control species to be higher than the control threshold S_{SPC}. It has been assumed that the concentration of species of interest C_{SOI} corresponds to the decision threshold SD. The objective is to determine the effect of varying the control threshold S_{SPC} on the sequencing duration for a sample in which the SOI is not detected.

TABLE 2

| | S_{SPC} | | | | |
|---|---|---|---|---|---|
| C_{ADN} | 5 | 25 | 125 | 625 | 3125 |
| 0.0001SD | 0:03 | 0:03 | 0:03 | 0:03 | 0:12 |
| 0.001SD | 0:03 | 0:03 | 0:03 | 0:03 | 0:12 |
| 0.01SD | 0:03 | 0:03 | 0:03 | 0:03 | 0:12 |
| 0.1SD | 0:03 | 0:03 | 0:03 | 0:03 | 0:12 |
| SD | 0:03 | 0:03 | 0:03 | 0:06 | 0:21 |
| 10SD | 0:03 | 0:03 | 0:06 | 0:21 | 1:45 |
| 100SD | 0:03 | 0:09 | 0:39 | 3:12 | 15:48 |
| 1000SD | 0:18 | 1:18 | 6:18 | 31:18 | 156:27 |
| 10000SD | 2:33 | 12:33 | 62:33 | 312:33 | 1562:42 |

A control threshold S_{SPC} of between 5 and 3125 is observed to be reached in less than 30 minutes of sequencing when the DNA concentration is equivalent to SD (C_{ADN}=SD). For these samples it is therefore possible to conclude the absence of SOI in less than 30 minutes of sequencing.

In instances in which the DNA concentration exceeds SD, the sequencing duration increases significantly, and it is therefore necessary to choose a control threshold which is not too high to allow a negative result to be returned for the detection of the species of interest SOI. For example, in the case of the clinical sample having an atypical infection with a concentration of the pathogen equivalent to 1000 SD, a control threshold S_{SPC}=125 does not allow a negative result to be returned for the detection of the species of interest SOI before at least 6 h of sequencing. It is therefore recommended that the threshold of the number of sequences S_{SOI} chosen is not too low to allow the sequencer to produce reads sufficiently to enable the detection of the SOI and to identify another species, different from the species of interest but the presence of which may nevertheless be of interest to report if its concentration is higher than SD. This situation may arise in the context of an atypical infection.

### Determining the Added Concentration of Control Species C_{SPC}

In the preceding paragraphs, the control species SPC was present at a concentration C_{SPC} equal to the decision threshold SD. The inventor has evaluated the effect of varying the added concentration C_{SPC} of the control species.

Table 3, for various concentrations of SPC added to the sample (C_{SPC}=0.01SD; C_{SPC}=SD and C_{SPC}=100SD), indicates the duration (hh:mm) needed to return a result and also the result of detection for various concentrations C_{SOI} of SOI of between 0.0001SD and 10 000 SD.

In the “Results” column:
The presence of concentrations expressed relative to the decision threshold SD in the “Results” columns indicates that the detection of reads of

$$SOI ( {N}_{SOI}^{i} > 0 )$$

and of

$$SPC ( {N}_{SPC}^{i} > 0 )$$

allowed this concentration to be calculated. This corresponds to step 62 or to step 64 with

$${N}_{SOI}^{i} > 0 . .$$

The entry “NEG” indicates that no read was associated with the

$$SOI ( {N}_{SOI}^{i} = 0 )$$

but that the number of reads associated with the SPC exceeds the control threshold set at 25

$$( {N}_{SPC}^{i} > 25 ) .$$

This corresponds to step 64 with

$${N}_{SOI}^{i} = 0 .$$

The entry “>SPC” indicates that no read was associated with the

$$SPC ( {N}_{SPC}^{i} = 0 )$$

but that the number of reads associated with the

$$SOI ( {N}_{SOI}^{i} > 1 \; 00 )$$

exceeds the threshold of the number of sequences S_{SOI}, set at 100. Under these conditions, it is not possible to calculate the concentration C_{SOI} of the species of interest, although this concentration may be deemed to be greater than the concentration of the control species C_{SPC}. This corresponds to step 63.

To start with, a low concentration C_{SPC}, equal to 0.01SD, was taken into account. When C_{SOI}>0,1SD, the durations found are substantially the same as in table 1. The added quantity of SPC therefore has no influence on the duration for reaching the threshold of the number of sequences S_{SOI}. However, the absence of reads of the SPC does not allow the concentration of SOI to be calculated, and the entry “>SPC” is not sufficient for comparison of this concentration relative to SD. When the concentration C_{SOI}<0,01SD, more than 12 hours of sequencing are needed in order to allow a result C_{SOI}<SD to be returned. In this context, if the total duration of sequencing is limited to 12 h, it is not possible to return negative results for these samples.

TABLE 3

| | C_{SPC} | | | | | |
|---|---|---|---|---|---|---|
| | 0.01SD | | SD | | 100SD | |
| C_{SOI} | TTR | Results | TTR | Results | TTR | Results |
| 0.0001SD | 12:33 | 0.0001SD | 0:09 | NEG | 0:03 | NEG |
| 0.001SD | 12:33 | 0.001SD | 0:09 | NEG | 0:03 | NEG |
| 0.01SD | 12:33 | 0.01SD | 0:09 | 0.008SD | 0:03 | NEG |
| 0.1SD | 5:03 | >SPC | 0:09 | 0.09SD | 0:03 | 0.05SD |
| SD | 0:33 | >SPC | 0:09 | SD | 0:03 | SD |
| 10SD | 0:06 | >SPC | 0:06 | 10SD | 0:03 | 10SD |
| 100SD | 0:03 | >SPC | 0:03 | 100SD | 0:03 | 100SD |
| 1000SD | 0:03 | >SPC | 0:03 | 1000SD | 0:03 | 1000SD |
| 10 000SD | 0:03 | >SPC | 0:03 | >SPC | 0:03 | 10 000SD |

A concentration C_{SPC} equal to the decision threshold increases the detection limit relative to a concentration C_{SPC}<0.01 SD. However, this allows the concentration of the species of interest to be quantified according to a greater dynamic: between 0.01SD and 1000SD.

Consideration was then given to a high concentration C_{SPC}, of 100 times the decision threshold SD. The high quantity of SPC allows a result to be returned very rapidly, since within the range of concentrations of SOI evaluated, the result is returned only 3 minutes after the start of the sequencing. However, an at least 10-fold increase in the detection limit is observed, relative to the tests carried out with a concentration C_{SPC}=SD. A possible explanation for this is that the high presence of SPC correspondingly reduces the number of reads associated with the SOI. Accordingly, the sensitivity is reduced.

The addition of a large quantity of SPC also allows a quantitative result to be returned over a wider range than for the lowest concentrations.

The various parameters of analysis may therefore be selected, such as:
the concentration C_{SPC} of the control species added to the sample;
the threshold of the number of sequences S_{SOI};
the control threshold of S_{SPC}.

The parameterization allows the performance levels in the method described above to be adapted in terms of rapidity of sequencing, quantification dynamics, and the required sensitivity.

For example, in the context of a clinical diagnosis requiring rapid detection with high sensitivity of detection and also precision in the quantification of the SOI detected around the clinical decision threshold, it is advantageous to add the SPC at a concentration close to the clinical decision threshold C_{SPC}≈SD (for example, C_{SPC} between 0.1SD and 10 SD or even between 0.01 SD and 100SD), with a threshold of the number of sequences S_{SOI} of between 5 and 500 and a control threshold S_{SPC} of between 5 and 125.

### Example 1

In a first example, Bacillus subtilis was used as a control species for the metagenomic sequencing of samples resulting from bronchoalveolar lavages (BALs) performed on human patients. This type of sample is known to be liable to comprise a substantial quantity of human DNA originating from the patient. 38 samples were sequenced. The maximum duration of sequencing was set at 12 hours. Each iteration comprised 4000 reads.

The volume of each sample was 600 μL. The control species was added at a concentration of 1.7E4 CFU/mL, with CFU signifying colony-forming unit(s). This threshold is deemed to be slightly above a clinical threshold which separates the normal presence of bacteria from a bacterial presence which is representative of an infection.

For removing the DNA from the patient, the analytical protocol includes the removal of the DNA from the patient in the course of a first lysis. In the first lysis, the sample was treated with a lyzing agent which specifically targets the cells of the patient. A lyzing agent of this kind is described for example in WO2014/114896. The DNA released was then removed by enzymatic action and washing. The sample then underwent a second, mechanical and chemical, lysis, to extract the bacterial DNA. The mechanical lysis was performed by means of stirring, lasting for 20 minutes, using glass beads 1 mm in diameter and also Zr/Si microbeads 0.1 mm in diameter. The DNA was extracted from the lysate using the easyMAG platform (Biomérieux). The volume resulting from the elution was 25 μL. The DNA extracts were stored at −20° C.

The preparation of the sequencing libraries and the barcoding of the DNA fragments were performed using the Rapid PCR Barcoding kit (Oxford Nanopore Technologies). 4 samples were treated in parallel. The sequencing was performed with the MinION sequencer (Oxford Nanopore Technologies), connected to a MinIT computer (Oxford Nanopore Technologies). The sequencing data were processed by the WIMP (What is in my Pot?) software from the suite EPI2ME (Oxford Nanopore Technologies). This software allows the sequences of each sample to be separated using barcodes (barcoding by molecular marker, with each sample being assigned a label enabling its identification), the identification of the bacterial or viral or fungal species corresponding to each sequence read, and the counting of the read number associated with each of the species identified.

Steps 40 to 70 were reiterated in series of 4000 reads. The detection thresholds respectively associated with the species of interest and with the control species were 100 reads and 25 reads. 20 different species of interest were taken into account: Escherichia coli, Klebsiella oxytoca, Klebsiella pneumoniae, Klebsiella aerogenes, Enterobacter cloacae, Serratia marcescens, Proteus mirabilis, Proteus vulgaris, Hafnia alvei, Citrobacter freundii, Citrobacter koseri, Morganella morganii, Providencia stuartii, Pseudomonas aeruginosa, Stenotrophomonas maltophilia, Acinetobacter baumannii, Legionella pneumophila, Haemophilus influenzae, Staphylococcus aureus and Streptococcus pneumoniae.

When the concentrations of each species of interest were quantified, the concentrations were compared to a metagenomic threshold of 5.53 GEq/mL. This is a decision threshold which allows the pathological presence of a species of interest in a sample resulting from BAL to be concluded.

The 38 samples were analyzed. When at least one species of interest SOI was detected, the median duration of sequencing was 28.5 minutes per sample. This corresponds to the duration of sequencing needed for the condition

$${N}_{SOI}^{i} \geq 100$$

to be obtained for at least one of the species of interest.

In the absence of detection of at least one of the species of interest

$$( {N}_{SOI}^{i} < 100 and {N}_{SPC}^{i} \geq 25 ) ,$$

the median duration of sequencing was 2 hours and 13 minutes. The condition

$${N}_{SPC}^{i} > {S}_{SPC}$$

makes it possible to ensure that sequencing has taken place appropriately. This allows an assurance that a negative sample is indeed a true negative.

The results of the sequencing according to the method described above were compared with sequencing of the same type, performed over a fixed duration of 12 hours. Table 4 summarizes the main results obtained. The performance levels of each technique were compared by analyzing each sample by the reference technique: microbial culture.

TABLE 4

| | 12-hour sequencing | Invention |
|---|---|---|
| True positives | 19 | 19 |
| False positives | 48 | 38 |
| False negatives | 4 | 4 |
| True negatives | 689 | 699 |
| Sensitivity | 82.6% | 82.6% |
| Specificity | 93.5% | 94.8% |
| Positive predictive value | 28.4% | 33.3% |
| Negative predictive value | 99.4% | 99.4% |

The sensitivity, specificity, positive predictive value (usually denoted PPV), and negative predictive value (usually denoted NPV) result from the number of the quantities of true positives, false positives, true negatives, and false negatives.

The method is observed to have performance levels equivalent to or even better than those obtained with a 12 h sequencing. It allowed all of the infections to be detected, with a median time gain of more than 11 hours and 30 minutes. Furthermore, in the absence of infection by the entirety of the species of interest, the detection of the SPC confirms the proper operation of the method in a median duration of 2 hours and 13 minutes and the production of a negative detection result.

4 false negatives are observed, but these detection failures are not attributable to the method described. 2 of the 4 false negatives were not detected by PCR, and it therefore appears that these are false culturing positives or errors in identification of the species by the reference technique. The other 2 false negatives correspond to the species S. marcescens, which produced a read number of less than 100 and which were quantified at concentrations lower than the clinical decision threshold. These 2 discordances between the method and the reference technique were confirmed by the PCR. The increase in the duration of sequencing to 12 hours does not improve the detection of these 2 infections with S. marcescens and suggests an error induced by the WIMP software used for assigning the sequences.

The method is found to enable improvement in the performance levels of the test, while lowering the number of false positives, thereby enhancing the specificity and the positive predictive value. This is due to the appropriate halting of sequencing when sufficient relevant information is available, namely at the time when

$${N}_{SOI}^{i} \geq {S}_{SOI}$$

or when

$${N}_{SPC}^{i} > {S}_{SPC} .$$

As soon as one of these thresholds is crossed, the iterations cease and the concentration (or minimum concentration) of the species of interest can be quantified. It is found not to be useful, or even desirable, to continue the iterations up to the maximum number of iterations initially set, corresponding to a total duration of 12 hours. The reason is that obtaining too large a number of sequences of the species of interest may give rise to false positives.

### Example 2

In a second example, the same protocol as described in connection with the first embodiment was used. 11 BAL samples were analyzed, prepared as described above. The 11 samples had been analyzed beforehand by bacterial culture and proved positive for at least one species of interest, with a concentration of more than 1^{E}4 CFU/mL. The 11 samples were analyzed so as to identify at least one of the 20 species of interest listed above. The iterative steps 40 to 80 were implemented. Of the 11 samples, 6 were deemed to be positive for at least one species of interest in less than 30 minutes of sequencing.

However, a quantity of 100 sequences for each species of interest detected is too low for achieving fine characterization of their genome. The iterative steps 100 to 120 were implemented on the samples, so as to identify the presence of possible antibiotic resistance markers in the genome. The iterative steps 100 to 120 were performed until a total duration of sequencing of 12 hours was obtained.

In table 5:
the first column (ref) corresponds to the reference of each sample;
the second column (SOI) corresponds to each species of interest detected;
the third column (technique) corresponds to the detection technique: culturing or implementation of the invention;
the fourth column (C_{SOI}) corresponds to the measured or estimated concentration of each bacterial species detected;
the fifth column (dep) corresponds to the depth of sequencing, as described above;
the sixth column (TTR ID) corresponds to the duration, in the format “hours:minutes” of steps 40 to 80, for confirmation of the presence of the species of interest in the sample. The duration is observed to be between 3 minutes (sample C1-026) and 8 hours 37 (sample C1-060). 6 samples were deemed to be positive in less than 30 minutes.
the following columns correspond to the presence of an antibiotic resistance marker in respect of 21 antibiotic agents. The letter R indicates a resistance observed to the antibiotic by culturing or predicted by the presence of a marker, while the letter S indicates a sensitivity observed by culturing. It is not possible to state an opinion on sensitivity to an antibiotic on the basis of the absence of markers for resistance to that antibiotic. This is the reason why there is no letter S in the lines corresponding to the implementation of the invention. A bold letter R indicates the presence of an antibiotic resistance marker revealed by the sequencing but not confirmed by bacterial culture (bold letter S): this is therefore a false positive resulting from the sequencing. A bold and larger font letter R indicates the presence of an antibiotic resistance marker revealed by bacterial culture but not detected by the sequencing: this is a false negative resulting from the sequencing. 9 false negatives are counted.

Of the 9 false negatives (bold and larger font letter R), 8 result from samples for which the sequencing depth is low, i.e., less than 30. It is therefore probable that the rate of false negatives can be reduced by continuing sequencing until a sequencing depth threshold is reached; in this example, this threshold may be equal to 30.

It is appreciated that a notable advantage of the invention lies in the possibility of obtaining increasingly precise information on the constitution of the sample as the iterations progress. When the task at hand is to determine the presence of species of interest, the invention allows the duration of analysis to be optimized, by stopping the iterations as soon as a biological species of interest is detected with sufficient reliability. The duration of analysis is therefore shortened, with no loss in sensitivity.

TABLE 5

| | | | | | TIR | Amino- | | | | |
|---|---|---|---|---|---|---|---|---|---|---|
| ref | SOI | technique | C_{SOI} | dep | ID | glycoside | Carbapenem | Cephalosporin | Cephamycin | Fluoroquinolone |
| C1-049 | E. coli | Culture | >1E+5 | | 0:07 | S | S | R | R | S |
| | | Invention | 1E+7 | 762.3 | | R | R | R | R | R |
| C1-052 | S. aureus | Culture | >1E+4 | | 0:07 | S | | | | S |
| | | Invention | 2E+6 | 494.1 | | | | | | |
| | E. coli | Culture | >1E+3 | | | S | S | S | S | R |
| | | Invention | 3E+2 | 0.1 | | | | | | |
| C1-022 | E. coli | Culture | >1E+5 | | 0:30 | S | S | R | R | S |
| | | Invention | 2E+7 | 2.0 | | | | R | | |
| C1-026 | H. influenzae | Culture | >1E+5 | | 0:03 | | | S | | |
| | | Invention | 6E+7 | 1433.3 | | | | | | |
| | K. pneumoniae | Culture | >1E+3 | | | | | | | |
| | | Invention | 2E+2 | 0.0 | | | | | | |
| | [symbol] | Culture | >1E+5 | | | | | S | | S |
| | | Invention | 5E+4 | 3.2 | | | | | | |
| C1-030 | H. influenzae | Culture | >1E+5 | | 0:03 | | | S | | |
| | | Invention | POS > MT | 229.2 | | | | R | | |
| | K. pnuemoniae | Culture | >1E+5 | | | S | S | S | S | S |
| | | Invention | POS > MT | 8.6 | | | R | R | | |
| C1-029 | S. aureus | Culture | >1E+4 | | 1:06 | S | | | | S |
| | | Invention | 2E+6 | 0.2 | | | | | | |
| C1-032 | P. aeruginosa | Culture | >1E+5 | | 3:44 | S | S | S | | S |
| | | Invention | 1E+4 | 0.0 | | | | | | |
| | [symbol] | Culture | >1E+4 | | | | | | | |
| | | Invention | 1E+6 | 1.6 | | | | | | |
| C1-035 | K. pnuemoniae | Culture | >1E+5 | | 0:56 | S | S | S | S | |
| | | Invention | 3E+5 | 2.4 | | | | R | | |
| C1-044 | S. pnuemoniae | Culture | >1E+5 | | 0:07 | | | S | | S |
| | | Invention | 1E+8 | 1040.4 | | | | | | |
| | H. influenzae | Culture | >1E+4 | | | | | S | | |
| | | Invention | 2E+6 | 17.3 | | | | | | R |
| C1-059 | S. aureus | Culture | >1E+5 | | 1:16 | S | | | | S |
| | | [symbol] | 2E+7 | 0.6 | | | | | | |
| | [symbol] | Culture | >1E+5 | | | | | S | | |
| | | Invention | 2E+8 | 17.2 | | | | | | S |
| | H. influenzae | Culture | >1E+3 | | | | | S | | |
| | | Invention | 4E+6 | 0.1 | | | | | | |
| C1-060 | H. influenzae | Culture | >1E+4 | | 8:37 | | | S | | |
| | | Invention | [symbol] | 0.0 | | | | | | R |
| | S. aureus | Culture | >1E+3 | | | | | | | |
| | | Invention | 6E+2 | 0.0 | | | | | | |
| | S. constelatus | Culture | 1E+3 | | | | | | | |
| | | Invention | 2E+4 | 0.0 | | | | | | |

| | | | | | | | | | Diaminopyrimidine/ |
|---|---|---|---|---|---|---|---|---|---|
| ref | SOI | technique | Fucidin | Furane | Glycopeptide | Lincosamide | Macrolide | Monobactam | Sulfonamide |
| C1-049 | E. coli | Culture | | S | | | | | S |
| | | Invention | | | | R | R | R | R |
| C1-052 | S. aureus | Culture | S | | | S | S | | S |
| | | Invention | | | | | | | |
| | E. coli | Culture | | S | | | | S | S |
| | | Invention | | | | R | R | | |
| C1-022 | E. coli | Culture | | | | | | S | |
| | | Invention | | | | | | R | |
| C1-026 | H. influenzae | Culture | | | | | | | |
| | | Invention | | | | | | | |
| | K. pneumoniae | Culture | | | | | | | |
| | | Invention | | | | | | | |
| | [symbol] | Culture | | | S | S | S | | |
| | | Invention | | | | R | R | | |
| C1-030 | H. influenzae | Culture | | | | | | | |
| | | Invention | | | | | | R | |
| | K. pnuemoniae | Culture | | | | | | S | S |
| | | Invention | | | | | | R | |
| C1-029 | S. aureus | Culture | | | | S | S | | S |
| | | Invention | | | | | | | |
| C1-032 | P. aeruginosa | Culture | | | | | | | R |
| | | Invention | | | | | R | | R |
| | [symbol] | Culture | | | | | | | |
| | | Invention | | | | | | | |
| C1-035 | K. pnuemoniae | Culture | | | | | | S | |
| | | Invention | | | | | | R | |
| C1-044 | S. pnuemoniae | Culture | | | S | S | R | | |
| | | Invention | | | | R | R | | |
| | H. influenzae | Culture | | | | | | | |
| | | Invention | | | | | | | |
| C1-059 | S. aureus | Culture | S | | | S | S | | S |
| | | [symbol] | | | | | | | |
| | [symbol] | Culture | | S | S | S | S | | |
| | | Invention | | | | | | | |
| | H. influenzae | Culture | | | | | | | |
| | | Invention | | | | | | | |
| C1-060 | H. influenzae | Culture | | | | | | | |
| | | Invention | | | | | | | |
| | S. aureus | Culture | | | | | | | |
| | | Invention | | | | | | | |
| | S. constelatus | Culture | | | | | | | |
| | | Invention | | | | | | | |

| | | | | Penam + | | Penem + | Peptide/ | | | | |
|---|---|---|---|---|---|---|---|---|---|---|---|
| ref | SOI | technique | Penam | inhibitor | Penem | inhibitor | Polymixin | Phosphomycin | Rifamycin | Steptogramin | Tetracycline |
| C1-049 | E. coli | Culture | R | R | R | S | S | S | | | S |
| | | Invention | R | R | R | R | R | R | R | | R |
| C1-052 | S. aureus | Culture | R | | | S | | S | S | S | |
| | | Invention | | | | | | R | | | R |
| | E. coli | Culture | R | S | R | R | S | R | | | S |
| | | Invention | R | R | R | R | | | | R | |
| C1-022 | E. coli | Culture | R | R | R | S | S | S | | | S |
| | | Invention | R | R | R | R | | | | | |
| C1-026 | H. influenzae | Culture | S | S | | | | | | | |
| | | Invention | | | | | | | | | |
| | K. pneumoniae | Culture | | | | | | | | | |
| | | Invention | | | | | | | | | |
| | [symbol] | Culture | S | | | | | | S | | R |
| | | Invention | | | | | | | | R | R |
| C1-030 | H. influenzae | Culture | R | S | | | | | | | |
| | | Invention | R | R | R | | | | | | |
| | K. pnuemoniae | Culture | R | S | R | S | S | S | | | S |
| | | Invention | R | | R | | | | | | |
| C1-029 | S. aureus | Culture | R | | | S | | | S | S | |
| | | Invention | | | | | | | | | R |
| C1-032 | P. aeruginosa | Culture | S | S | S | | | | | | |
| | | Invention | | | | | | | | | R |
| | [symbol] | Culture | R | | | | | | | | |
| | | Invention | | | | | | | | | |
| C1-035 | K. pnuemoniae | Culture | R | S | R | | S | | | | S |
| | | Invention | R | | R | | | | | | |
| C1-044 | S. pnuemoniae | Culture | S | | | | | | S | S | S |
| | | Invention | | | | | | | | R | R |
| | H. influenzae | Culture | R | R | | | | | | | |
| | | Invention | | | | | | | | | |
| C1-059 | S. aureus | Culture | R | | | S | | S | S | | |
| | | [symbol] | R | | | | | | | | R |
| | [symbol] | Culture | S | | | | | S | S | S | S |
| | | Invention | | | | | | | | | R |
| | H. influenzae | Culture | R | S | | | | | | | |
| | | Invention | | | | | | | | | |
| C1-060 | H. influenzae | Culture | S | S | | | | | | | |
| | | Invention | | | | | | | | | |
| | S. aureus | Culture | | | | | | | | | |
| | | Invention | | | | | | | | | |
| | S. constelatus | Culture | | | | | | | | | |
| | | Invention | | | | | | | | | |

[symbol]

indicates data missing or illegible when filed

As shown in example 2, following detection of a species of interest, complementary iterations may be performed, to obtain more precise information on the genome of each species of interest detected.

Despite being described with sequencing performed by means of a MinION sequencer (Oxford Nanopore Technologies), the invention may be implemented by means of other types of sequencers allowing the reading and the real-time analysis of the fragments of DNA present in a sequencing library or directly in the sample.

A method has been described in which the steps following sequencing are implemented by a processing unit connected to the sequencer, in particular a unit based on one or more processors or microprocessors and having a computer memory which memorizes all of steps 40 to 130 in the form of instructions which can be executed by computer, the memory being configured to memorize the results of the method and the values, parameters and intermediate results. This unit is advantageously connected to a screen for displaying results of the method according to the invention for the attention of the user; this screen may constitute the screen of a mobile device (e.g., smartphone) connected to the processing unit for receiving and displaying the results. These steps may be implemented remotely, for example on a remote server connected to the sequencers, or directly, or through a laboratory computing system. These steps may also be implemented on a mobile device (e.g., smartphone, tablet) connected to the sequencer, for example a MinION, with the assembly consisting of the MinION and the mobile device being readily transportable. These steps may also be implemented by different processing units. For example, one unit, connected to the sequencer, implements the bioinformatic steps (signal reading, translation into nucleotide bases, assemblies, quality control, etc.) and another processing unit, connected to the first, implements the rest of the method.

1. A method for detecting a biological species of interest in an analysis sample, the biological species of interest having a known or partially known genome, the sample comprising a mixture of different biological species, the method comprising:
a) adding a control species to the sample, the control species having a known genome, the control species being added in a known concentration to the sample;
b) extracting nucleic acids from the sample;
c) sequencing a portion of the nucleic acid sequences extracted in the extracting b), using a real-time sequencer, for a predetermined duration or until a predetermined number of sequencings is obtained;
d) after the sequencing c), assigning sequences resulting from the sequencing c) to the species of interest and to the control species;
e) updating quantities of sequences respectively associated with the species of interest and the control species, so that the quantities of sequences associated with the control species and with the species of interest are:
in a first iteration of the updating e), the quantities of sequences assigned to the control species and to the species of interest in the assigning d), added to quantities of initial sequences;
in each reiteration of the updating e), the quantities of sequences assigned to the control species and to the species of interest in the assigning d), added to the quantities of sequence associated with the control species and with the species of interest resulting from the preceding iteration;
f) comparing
the quantity of sequence associated with the species of interest updated in the updating e) with a threshold of the number of sequences,
and/or
the quantity of sequence associated with the control species updated in the updating e) with a control threshold;
g) depending on at least one comparison made in the comparing f), detecting the presence of the biological species of interest in the sample or proceeding to a reiterating h); and
h) reiterating the sequencing c), assigning d), updating e), comparing f) and detecting or proceeding g) until a first iteration stop criterion is reached.

2. The method as claimed in claim 1, wherein the assigning d) of each iteration is carried out for a predetermined duration or until a predetermined number of sequences are sequenced.

3. The method as claimed in claim 1, wherein:
in the comparing f) of an iteration, when the quantity of sequences associated with the species of interest is greater than the threshold of the number of sequences;
in the updating e) of the iteration, when the quantity of sequences associated with the control species is strictly greater than zero;
and wherein the detecting g) of the iteration comprises an estimation of a concentration of the species of interest, depending:
on the quantity of sequences associated with the species of interest at the last updating;
on the quantity of sequences associated with the control species at the last updating;
on the added concentration of the control species.

4. The method as claimed in claim 1, wherein:
in the comparing f) of an iteration, when the quantity of sequences associated with the species of interest is greater than the threshold of the number of sequences;
in the updating e) of the iteration, when the quantity of sequences associated with the control species is zero;
during the detecting or proceeding g) of the iteration, the concentration of the species of interest is deemed to be greater than the concentration of the control species.

5. The method as claimed in claim 1, wherein:
in the comparing f) of an iteration, when the quantity of sequences associated with the species of interest is less than the threshold of the number of sequences
in the comparing f) of the iteration, when the quantity of sequences associated with the control species is greater than the control threshold;
during the detecting or proceeding g) of the iteration, if the quantity of sequences associated with the species of interest is zero, the concentration of the species of interest is deemed to be zero.

6. The method as claimed in claim 1, wherein:
in the comparing f) of an iteration, when the quantity of sequences associated with the species of interest is less than the threshold of the number of sequences;
in the comparing f) of the iteration, when the quantity of sequences associated with the control species is greater than the control threshold;
during the detecting or proceeding g) of the iteration, if the quantity of sequences associated with the species of interest is non-zero, the detecting or proceeding g) comprises an estimation of a concentration of the species of interest, depending:
on the quantity of sequences associated with the species of interest;
on the quantity of sequences associated with the control species;
on the added concentration of the control species.

7. The method as claimed in claim 3, wherein the concentration of the species of interest is estimated from a ratio:
$$\frac{{N}_{SOI}^{i}}{{N}_{SPC}^{i}} \times \frac{{L}_{SPC}}{{L}_{SOI}} \times {C}_{SPC}$$
in which:
L_{SPC} and L_{SOI} are respectively genome lengths of the control species and of the species of interest;
$${N}_{SOI}^{i} and {N}_{SPC}^{i}$$
are respectively the quantities of sequences respectively associated with the species of interest and with the control species resulting from the last updating; and
C_{SPC} is the concentration of the control species added to the sample.

8. The method as claimed in claim 1, wherein after the iteration stop criterion has been reached,
if the quantity of sequences associated with the species of interest is less than the threshold of the number of sequences;
and if the quantity of sequences associated with the control species is less than the control threshold;
the method generates information indicating that neither the presence nor the absence of the species of interest can be confirmed.

9. The method as claimed in claim 1, comprising taking into account a decision threshold, the method aiming to compare a concentration of the species of interest to the decision threshold, the method being so that in the adding a), the concentration of the control species is in a range of from 0.01 times to 100 times the decision threshold.

10. The method as claimed in claim 1, wherein in the reiterating h), the iteration stop criterion is reached when at least one of the following conditions is met:
the quantity of sequences associated with the species of interest exceeds the threshold of the number of sequences;
the quantity of sequences associated with the control species exceeds the control threshold;
the cumulative duration of the sequencing c) reaches a predetermined maximum duration;
the cumulative number of sequencings in the sequencings c) reaches a predetermined maximum number;
the number of iterations reaches a predetermined maximum number of iterations.

11. The method as claimed in claim 1, wherein following detection of the presence of the species of interest in the sample, the method comprises:
i) sequencing a portion of the nucleotide sequences extracted in the extracting b), using a real-time sequencer, for a predetermined duration or until a predetermined number of sequencings is obtained;
j) following the sequencing i), assigning sequences resulting from the sequencing i) to the species of interest detected;
k) updating the quantities of sequences associated with the species of interest, so that the quantities of sequences associated with the species of interest are:
in a first iteration of the updating k), the quantity of sequences assigned to the species of interest detected in the assigning j), added to the quantity of sequences resulting from the last iteration of the sequencing c), assigning d), updating e), comparing f) and detecting or proceeding g);
in each reiteration if the updating k), the quantity of sequences assigned to the species of interest in the assigning j), added to the quantity of sequences resulting from the preceding iteration of the updating k);
l) reiterating the sequencing i), assigning j), and updating k) until a second iteration stop criterion is reached.

12. The method as claimed in claim 11, wherein the sequencing i), assigning j), and updating k) are reiterated until a predetermined number of iterations is reached.

13. The method as claimed in claim 12, wherein the method comprises:
during each iteration of the sequencing i), assigning j), and updating k), following the updating k), determining a depth of sequencing of the biological species of interest, the depth of sequencing corresponding to a ratio between the cumulative length of the sequences associated with the species and the length of the genome of the species; and
reiterating the sequencing i), assigning j), and updating k) until a predetermined depth of sequencing is reached.

14. The method as claimed in claim 11, comprising, following stopping of the iterations of the sequencing i), assigning j), and updating k), detecting a typical sequence of the genome of the species of interest, the typical sequence comprising an antibiotic resistance marker or a virulence marker for the species of interest.

15. The method as claimed in claim 1, wherein the sequencer is configured to carry out successive sequencings of different sequences of nucleic acids, one after another, the sequencing of each sequence being deemed to be carried out in real time.

16. A device for metagenomic analysis of a sample, comprising:
a fluidic chamber intended to receive the sample;
a sequencer configured to carry out real-time sequencing of the sample;
a processing unit, comprising a bioinformatics module programmed to assign sequences resulting from the sequencer to a species, and a control module programmed to implement;
c) sequencing a portion of the nucleic acid sequences extracted in the extracting b), using a real-time sequencer, for a predetermined duration or until a predetermined number of sequencings is obtained;
d) after the sequencing c), assigning sequences resulting from the sequencing c) to the species of interest and to the control species;
e) updating quantities of sequences respectively associated with the species of interest and the control species, so that the quantities of sequences associated with the control species and with the species of interest are:
in a first iteration of the updating e), the quantities of sequences assigned to the control species and to the species of interest in the assigning d), added to quantities of initial sequences;
in each reiteration of the updating e), the quantities of sequences assigned to the control species and to the species of interest in the assigning d), added to the quantities of sequence associated with the control species and with the species of interest resulting from the preceding iteration;
f) comparing
the quantity of sequence associated with the species of interest updated in the updating e) with a threshold of the number of sequences,
and/or
the quantity of sequence associated with the control species updated in the updating c) with a control threshold:
g) depending on at least one comparison made in the comparing f), detecting the presence of the biological species of interest in the sample or proceeding to a reiterating h); and
h) reiterating the sequencing c), assigning d), updating c), comparing f) and detecting or proceeding g) until an iteration stop criterion is reached.

17. A recording medium, readable by computer or downloadable, comprising instructions for implementing;
c) sequencing a portion of the nucleic acid sequences extracted in the extracting b), using a real-time sequencer, for a predetermined duration or until a predetermined number of sequencings is obtained:
d) after the sequencing c), assigning sequences resulting from the sequencing c) to the species of interest and to the control species;
c) updating quantities of sequences respectively associated with the species of interest and the control species, so that the quantities of sequences associated with the control species and with the species of interest are:
in a first iteration of the updating c), the quantities of sequences assigned to the control species and to the species of interest in the assigning d), added to quantities of initial sequences;
in each reiteration of the updating c), the quantities of sequences assigned to the control species and to the species of interest in the assigning d), added to the quantities of sequence associated with the control species and with the species of interest resulting from the preceding iteration;
f) comparing
the quantity of sequence associated with the species of interest updated in the updating c) with a threshold of the number of sequences,
and/or
the quantity of sequence associated with the control species updated in the updating c) with a control threshold;
g) depending on at least one comparison made in the comparing f), detecting the presence of the biological species of interest in the sample or proceeding to a reiterating h); and
h) reiterating the sequencing c), assigning d), updating c), comparing f) and detecting or proceeding g) until an iteration stop criterion is reached.

18. The method as claimed in claim 2, wherein:
in the comparing f) of an iteration, when the quantity of sequences associated with the species of interest is greater than the threshold of the number of sequences;
in the updating e) of the iteration, when the quantity of sequences associated with the control species is strictly greater than zero;
and wherein the detecting g) of the iteration comprises an estimation of a concentration of the species of interest, depending:
on the quantity of sequences associated with the species of interest at the last updating;
on the quantity of sequences associated with the control species at the last updating;
on the added concentration of the control species.

19. The method as claimed in claim 2, wherein:
in the comparing f) of an iteration, when the quantity of sequences associated with the species of interest is greater than the threshold of the number of sequences;
in the updating e) of the iteration, when the quantity of sequences associated with the control species is zero;
during the detecting or proceeding g) of the iteration, the concentration of the species of interest is deemed to be greater than the concentration of the control species.

20. The method as claimed in claim 6, wherein the concentration of the species of interest is estimated from a ratio:
$$\frac{{N}_{SOI}^{i}}{{N}_{SPC}^{i}} \times \frac{{L}_{SPC}}{{L}_{SOI}} \times {C}_{SPC}$$
in which:
L_{SPC} and L_{SOI} are respectively genome lengths of the control species and of the species of interest;
$${N}_{SOI}^{i} and {N}_{SPC}^{i}$$
are respectively the quantities of sequences respectively associated with the species of interest and with the control species resulting from the last updating; and
C_{SPC} is the concentration of the control species added to the sample.
